(19) United States
(12) Patent Application Publication
(10) Pub. No.: US 2013/0261379 A1
(43) Pub. Date: Oct. 3, 2013

(54) Devices and Methods for the Treatment of Obesity

(71) Applicant: Theodore M. BENDER, San Anselmo, CA (US)

(72) Inventor: Theodore M. BENDER, San Anselmo, CA (US)

(73) Assignee: VIBRYNT, INC., Redwood City, CA (US)

(21) Appl. No.: 13/437,615

(22) Filed: Apr. 2, 2012

(51) Int. Cl. A61B 17/122; A61F 2/00

(52) U.S. Cl. 600/37

(57) ABSTRACT

A device includes an expandable body having a helical configuration. The body further includes expandable regions that contact a comparatively high surface area of the stomach as compared to their expanded volume. Methods for implanting the device and for treating a patient are provided.

### BACKGROUND OF THE INVENTION

Embodiments of the present invention relate generally to medical devices and methods and more particularly to minimally invasive devices, systems and methods for treating obesity.

Obesity has become a major health concern, both nationally and internationally. The National Center for Health Statistics (NCHS) estimates that over 120 million Americans are overweight, including about 56% of the adult population. Of these, about 52 million are considered obese, as measured by a body mass index (BMI) of 30% or greater. In Europe, an estimated 77 million people are obese, as measured by the same standard. This problem is not limited to western nations, as many developing countries are reported to have obesity rates over 75% of the adult population.

Co-morbidities that are associated with obesity include, but are not limited to type II Diabetes, high blood pressure, sleep apnea, stroke and arthritis, the symptoms of which often tend to be lessened or alleviated upon loss of weight by a person so affected.

In the U.S., options for treatment of obesity are currently quite limited. Current treatment methodologies typically rely upon surgically introducing a “malabsorptive” environment in the gastro-intestinal tract, a restrictive environment, or a combination of these. One available treatment method is gastric bypass surgery and another is referred to as gastric banding (one of these techniques if referred to as the LAPBAND™ procedure). These procedures are limited to only those patients with a BMI over 40 (or over 35, with co-morbidities present).

Gastric bypass procedures incur a great deal of morbidity and create a malabsorptive state in the patient by bypassing a large portion of the intestines. Serious side effects, such as liver failure have been associated with this procedure, as well as chronic diarrhea. Another surgical procedure that has a high degree of morbidity associated with it is known as the “Gastric Bypass Roux-en-Y” procedure. This procedure reduces the capacity of the stomach by creating a smaller stomach pouch. The small space holds only about one ounce of fluid. A tiny stomach outlet is also surgically created to slow the speed at which food leaves the stomach. Staples are used to create a small (15 to 20 cc) stomach pouch, with the rest of the stomach being stapled completely shut and divided from the stomach pouch. The small intestine is divided just beyond the duodenum, brought up, and connected to the newly formed stomach pouch. In addition to the considerable morbidity associated with this procedure, other disadvantages include “dumping syndrome”, where stomach contents are literally “dumped” rapidly into the small intestine which may lead to nausea, weakness, sweating, faintness, and diarrhea; hernias resulting from the surgery; gallstones; leakage of the connection between the pouch and the intestine; stretching of the pouch that was formed; nutritional deficiencies; and possible dehiscence of the staples.

The LAPBAND™ is a band that, when placed, encircles the fundus-cardia junction and is inflatable to constrict the same. It does not reduce the volume of the stomach, but rather restricts passage of food into the stomach, the theory being that the patient will feel satiety with a much less volume of food than previously. Although the LAPBAND™ procedure is less invasive than a gastric bypass procedure, it also typically achieves less weight loss. Further, it is not a simple procedure and requires a substantial amount of training by a surgeon to become proficient in performing the procedure. Also, a substantial amount of dissecting and suturing is required because the pathway by which the band is introduced is not an existing pathway, and must be established by dissection. Great care is required to avoid blood vessels and nerves that may be in the intended pathway to be created by the dissection. After placing the band around the fundus-cardia junction, the ends of the band must be connected together and then it must be cinched down into place. Additionally, complications such as erosion at the fundus-cardia junction, slippage of the band from its intended location, nausea/vomiting, gastroesophageal reflux, dysphagia and lack of effectiveness in causing weight loss have been reported.

Intra-gastric balloons have also been placed, in an attempt to fill a portion of the volume in the stomach, with the theory being that it will then require less food than previously, to give the patient a sensation of fullness or satiety. This procedure involves delivery of a balloon (typically, trans-orally) to the interior of the stomach and inflation of the balloon to take up a portion of the volume inside the stomach. However, intra-gastric balloons may also lead to complications such as obstruction, vomiting and/or mucosal erosion of the inner lining of the stomach. The balloon can break down over extended exposure to the stomach's acids, and in some cases, after breaking down, the balloon translated through the intestines and caused a bowel obstruction.

Gastrointestinal sleeves have been implanted to line the stomach and/or a portion of the small intestines to reduce the absorptive capabilities of the small intestine and/or to reduce the volume in the stomach, by reducing the available volume to the tubular structure of the graft running therethrough. Although weight loss may be effective while these types of devices are properly functioning, there are complications with anchoring the device within the stomach/GI tract, as the stomach and GI tract function to break down things that enter into them and to move/transport them through. Accordingly, the integrity of the anchoring of the device, as well as the device itself may be compromised over time by the acids and actions of the stomach and GI tract.

A sleeve gastrectomy is an operation in which the left side of the stomach is surgically removed. This results in a much reduced stomach which is substantially tubular and may take on the shape of a banana. This procedure is associated with a high degree of morbidity, as a large portion of the stomach is surgically removed. Additionally, there are risks of complications such as dehiscence of the staple line where the staples are installed to close the surgical incisions where the portion of the stomach was removed. Further, the procedure is not reversible.

In the laparoscopic duodenal switch, the size of the stomach is reduced in similar manner to that performed in a sleeve gastrectomy. Additionally, approximately half of the small intestine is bypassed and the stomach is reconnected to the shortened small intestine. This procedure suffers from the same complications as the sleeve gastrectomy, and even greater morbidity is associated with this procedure due to the additional intestinal bypass that needs to be performed. Still further, complications associated with malabsorption may also present themselves.

An inflatable gastric device is disclosed in U.S. Pat. No. 4,246,893, in which a balloon is inserted anteriorly of the stomach and posteriorly of the left lobe of the liver. The balloon is then inflated to compress the stomach so that it fills with less food that would ordinarily be possible. Not only does this device compress the stomach, but it also compresses the liver, as seen in FIG. 5 of the patent, which may cause complications with the liver function. Additionally, the balloon is simply placed into this location, and there is no assurance that it will not migrate and lose its effectiveness in compressing the stomach to the degree intended. Still further, the balloon is of a simple spherical design, and, as such, extends pressure outwardly in all directions, 360 degrees in all planes. Accordingly, the liver is compressed just as much as the stomach is. Also, the compression forces against the stomach are not ideal, as the spherical balloon conformation does not match the conformation of the expanding stomach. The stomach is not spherical when expanded, or concave with a constant radius of curvature, but expands into a designated space that allows the fundus to expand preferentially more than other parts of the stomach.

Brazzini et al. in WO2005/18417 discloses at least two or more expandable devices used to treat obesity, in which the devices are inserted through the abdominal wall and anchored subcutaneously or to the stomach wall to exert pressure against the external surface of the stomach wall.

U.S. Patent Publication No. 2005/0261712 to Balbierz et al. describes capturing a device against the outer surface of the stomach wall to form a restriction that appears to function similarly to the restriction imposed by the LAPBAND™. The anchoring of the devices disclosed relies upon placement of features against the internal wall of the stomach to form an interlock with the device which is placed against the external wall of the stomach.

U.S. Patent Publication No. 2005/0267533 to Gertner discloses devices for treatment of obesity that use one or more anchoring mechanisms that are passed through the wall of the stomach to establish an anchor. The stomach is reduced in size by passing the devices through the stomach wall on opposite sides of the stomach and compressing the walls together to eliminate a portion of the interior space within the stomach. Gertner also discloses an embodiment in which an extra-gastric balloon is placed anteriorly of the stomach and attached to the abdominal wall using one of the anchoring mechanisms described.

U.S. Pat. No. 6,981,978 to Gannoe discloses devices for reducing the internal cavity of the stomach to a much smaller volume, which may be used to carry out a bypass procedure. Stapling is employed to isolate the smaller volume in the stomach, and thus the same potential disadvantages are present as with other stapling procedures described herein.

U.S. Pat. No. 6,186,149 to Pacella et al. describes an occluder device that can be used as a dietary control device (see FIG. 8C). The occluder device is placed against the wall of the stomach and inflated to press inwardly on the stomach wall. A frame is wrapped around the stomach wall and is inflated to press against the stomach wall. However, there is no disclosure of how the frame might be adjusted to maintain a position relative to the stomach wall as the size of the stomach varies.

Gastric reduction techniques have been attempted, such as by inserting instruments trans-orally and reducing the volume of the stomach by stapling portions of it together. However, this technique is prone to failure due to the staples pulling through the tissues (i.e., dehiscence) that they are meant to bind.

Techniques referred to as gastric pacing endeavor to use electrical stimulation to simulate the normal feedback mechanisms of a patient that signal the brain that the patient is full, or satiated. While these techniques are less invasive than some of the other existing treatments, statistics to date have shown that the amount of weight lost by using such techniques is less than satisfactory.

Currently marketed drugs for weight loss, such as XENICAL®, MERIDIA® and Phen fen have largely failed, due to unacceptable side effects and complications, and sometimes to an ineffective amount of weight loss. Other drugs that are on the horizon include ACCOMPLIA® and SYMLIN®, but these are, as yet, unproven.

Methods that places devices such as bands, balloons, or wraps on the extra-gastric surface of the stomach carry the risk of creating erosions on the stomach surface. Such erosions can lead to migration of the device, even through to the stomach. Erosions carry the risk of spillage of stomach content into the abdominal cavity, which can be a life-threatening event. Erosions can be caused by sharp edges or focused pressure on the stomach wall. Constriction of the stomach applies force to the stomach wall and devices that do so can create erosions.

The risk and invasiveness factors of currently available surgeries are often too great for a patient to accept to undergo surgical treatment for his/her obesity. Accordingly, there is a need for less invasive, yet effective surgical treatment procedures for morbidly obese patients (patients having a BMI of 35 or greater). Also, since the current surgical procedures are currently indicated only for those patients having a BMI of 40 or greater, or 35 or greater when co-morbidities are present, it would be desirable to provide a surgical procedure that would be available for slightly less obese patients, e.g., patients having a BMI of 30 to 35 who are not indicated for the currently available surgical procedures. It would further be desirable to provide a surgical procedure that would be indicated for obese patients having a BMI in the range of 30-35, as well as for more obese patients.

There is a need for devices and methods for treating obesity with minimally invasive devices that substantially preserve the physiological integrity of the blood vessels around the stomach while providing an adjustable, constrictive force on the stomach.

### BRIEF SUMMARY OF THE INVENTION

Certain embodiments of the present invention are related to a device for the treatment of obesity. The device includes an expandable body having a helical configuration suitable for being positioned about the exterior of the stomach of a patient. The expandable body includes a first region having an elongated configuration when expanded and a second region having a lobed configuration when expanded.

Certain embodiments of the present invention are related to a method for the treatment of obesity. The method includes accessing an extra-gastric abdominal space of a patient and introducing an expandable device into the extra-gastric abdominal space. The expandable device has a first region having an elongated configuration when expanded and a second region having a lobed configuration when expanded. The expandable device has a helical configuration suitable for being positioned about the exterior of the stomach. The method further includes advancing the expandable device around the stomach and expanding the first region and the second region to constrain the stomach.

Certain embodiments of the present invention are related to a device for the treatment of obesity. The device includes an expandable body having a helical configuration suitable for being positioned about the exterior of the stomach of a patient. The body has at least one expandable region having surface area to internal volume ratio of greater than about 1.5:1 when expanded.

### BRIEF DESCRIPTION OF SEVERAL VIEWS OF THE DRAWINGS

FIG. 1 illustrates the stomach, several of the blood vessels, and the connective tissue present near the stomach.

FIG. 2 illustrates a perspective view of a device according to certain embodiments of the invention.

FIG. 3 illustrates a perspective view of a device having a reinforced region according to certain embodiments of the invention.

FIG. 4A illustrates a cross-sectional view of a reinforced region of the device of FIG. 3.

FIGS. 4B and 4C illustrate alternative embodiments of cross-sections of reinforced regions of devices according to certain embodiments of the invention.

FIG. 5 illustrates a perspective view of a device having a reinforced region according to certain embodiments of the invention.

FIG. 6 illustrates a perspective view of a device having a lobed region according to certain embodiments of the invention.

FIG. 7 illustrates a perspective view of a device having a plurality of lobed regions according to certain embodiments of the invention.

FIGS. 8A through 8D illustrate plan views of various configurations of lobed regions according to certain embodiments of the invention.

FIG. 9A illustrates a cross-sectional view of the lobed region of FIG. 8C.

FIG. 9B illustrates a cross-sectional view of a lobed region having a reinforced region according to certain embodiments of the invention.

FIG. 10 illustrates a cross-sectional view of a device in which an unexpanded lobed region is wrapped about a region of the device according to certain embodiments of the invention.

FIG. 11 illustrates a perspective view of a device being advanced around the stomach according to certain embodiments of the invention.

FIG. 12 illustrates a perspective view a device being advanced around the stomach such that a region of the device is positioned near the fundus according to certain embodiments of the invention.

FIG. 13 illustrates a perspective view of regions of a device being expanded after the device is positioned about the stomach according to certain embodiments of the invention.

FIG. 14 illustrated a perspective view of a device compressing a stomach according to certain embodiments of the invention.

### DETAILED DESCRIPTION OF THE INVENTION

Embodiments of the present invention are useful in the surgical treatment of obesity. The description, figures, and examples herein relate to devices and methods for the containment or constriction of the stomach.

Before the present devices and methods are described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

Short summaries of certain terms are presented in the description of the invention. Each term is further explained and exemplified throughout the description, figures, and examples. Any interpretation of the terms in this description should take into account the full description, figures, and examples presented herein.

The singular terms “a,” “an,” and “the” include plural referents unless the context clearly dictates otherwise. Thus, for example, reference to an object can include multiple objects unless the context clearly dictates otherwise. Similarly, references to multiple objects can include a single object unless the context clearly dictates otherwise.

The terms “helix,” “helical,” and the like refer to a three-dimensional curve with generally continuous curvature in which the curve turns about an axis in a cylindrical or conical fashion while rising at a generally constant upward angle from a base. The helix can be right-handed or left-handed and need not turn about the axis at a perfectly constant or constantly varying distance from the axis.

The terms “lobe,” “lobed,” and the like refer to a rounded or blunt projection. The extent of roundedness or bluntness can vary among lobes or lobed shapes and it is understood that the term “rounded” does not limit the shape of the lobe to circular or spherical projections.

The terms “expand,” “expanded,” “expandable,” “expanding,” “expansion,” and the like refer to an object or region of an object becoming larger in size or volume. In certain cases herein, such expansion is accomplished by at least partially filling an internal volume of the object or region. However, it is understood that these terms include cases where an internal volume of the object or region is filled but the object or region does not stretch or grow. In such cases where filling the object does not cause the object or region to stretch or grow, the act of at least partially filling the internal volume of the object or region is the act of expansion. Conversely, the term “unexpanded” and the like refers to both the condition of an object or a region prior to it becoming larger in size or volume and to the condition of an object or region prior to its internal volume being at least partially filled.

The terms “substantially,” “substantial,” and the like refer to a considerable degree or extent. When used in conjunction with an event or circumstance, the terms can refer to instances in which the event or circumstance occurs precisely as well as instances in which the event or circumstance occurs to a close approximation, such as accounting for typical tolerance levels or variability of the embodiments described herein.

A “compliant” material refers to a material that is stretchable or expandable. This expansibility allows the material to increase in dimension substantially more than a noncompliant or semi-compliant material, prior to failure. For example, when formed as an expandable body, a compliant material comprises an expansibility property of being able to increase its radius, beyond its formed radius, under pressure applied into the expandable body, by 100 percent or more, without rupturing.

A “noncompliant” material refers to a material that, when formed as an expandable body, can increase its radius beyond its formed radius, under pressure applied into the expandable body, only up to about 10 percent or less prior to rupturing.

A “semi-compliant” material refers to a material that, when formed as an expandable body, can increase its radius beyond its formed radius, under pressure applied into the expandable body, by an amount between about 10 percent and about 100 percent, prior to rupturing.

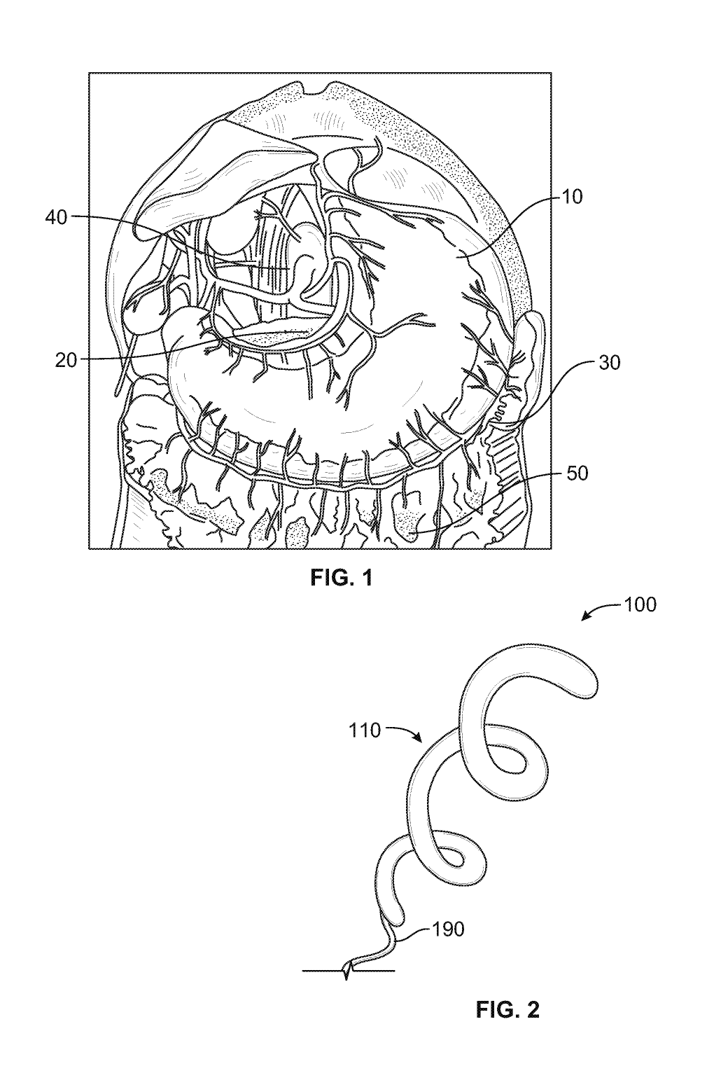

FIG. 1 illustrates stomach 10, several of the blood vessels, and the connective tissue present near stomach 10. Some of these blood vessels supply blood to the stomach itself and other blood vessels supply blood to internal organs such as the spleen or the liver. For example, located along or nearby the inner curvature 20 (or lesser curvature) of the stomach are the left gastric artery, the celiac trunk, the common hepatic artery, the aorta, and the right gastric artery. Other smaller branch arteries emanate from some of these arteries, making the inner curvature 20 an area rich with blood vessels important for the function of abdominal organs. Similarly, located along or nearby the outer curvature 30 (or greater curvature) of the stomach are the right and left gastroepoploic arteries, sections of the splenic artery, and a series of short gastric arteries. As compared to inner curvature 20, outer curvature 30 has a lower density of blood vessels, both those that supply the stomach 10 and those that supply other organs or muscles. In abdominal surgeries that involve the stomach, the area around the outer curvature tends to tolerate surgical dissection more readily than the area around the inner curvature in part because of the lower density of blood vessels in the area around the outer curvature.

Referring still to FIG. 1, connective tissue is present in the areas of both inner curvature 20 and outer curvature 30. Lesser omentum 40 is present in the area around inner curvature 20 and greater omentum 50 is present in the area around outer curvature 30. As used herein, “omentum” refers to lesser omentum 40, greater omentum 50, or both as the context dictates.

One of the novel aspects of the present invention is an appreciation of the importance of substantially maintaining the integrity of the blood vessels and connective tissue on or near both the inner curvature and the outer curvature of the stomach while still providing an effective treatment for obesity. Unlike devices and methods of the prior art that often depict and treat the stomach as a freestanding organ with little to no regard for the important blood vessels attached to or nearby it, certain embodiments of the present invention are configured to conform with the actual anatomy of the stomach and its milieu. Unlike devices and methods of the prior art, certain embodiments of the present invention are configured to facilitate the preservation of many of these blood vessels and much of the connective tissue both during the initial implantation of the device and during the lifetime of the device.

An advantage of certain devices and methods of the present invention as described in detail herein is that stomach 10 can be constricted or constrained without the blood supply to the stomach being cut off due to compression or dissection of blood vessels. As compared to some prior art instances of stomach constriction or constraint, the design, configuration, and execution of devices and methods of certain embodiments of the invention substantially avoid compressing or dissecting major blood vessels by allowing parts of the device to be placed away from the blood vessels yet also to apply and to maintain the necessary constrictive and/or constraining conditions. The advantage of preserving the blood vessels to the stomach is that it reduces the likelihood of the treatment cutting off blood flow (to and from the stomach) to a section of the stomach, which can lead to ischemia, which can cause leaks in the stomach, which can lead to an emergency surgery where much of the stomach may need to be removed. An advantage to preserving the connective tissues to the stomach is that the connective tissues help maintain the position of the stomach, and reduce the likelihood of the stomach twisting or curling up on itself, as can happen if the connective tissues are removed. Thus preservation of the vessels and connective tissue helps to avoid serious medical complications that are associated with bariatric surgeries that do not preserve these tissue connections.

Similarly, an advantage of certain devices and methods of the present invention as described in detail herein is that stomach 10 can be constricted or constrained without complete dissection of the vessels or connective tissue near stomach 10. Advantageously, the presence of substantially intact connective tissue can help devices of certain embodiments of the invention maintain the necessary constrictive and/or constraining conditions.

Another advantage of certain devices and methods of the present invention as described in detail herein is the relatively wide distribution of constrictive or constraining forces about stomach 10. The relatively high level of surface contact of certain embodiments of the device with the stomach allows for constrictive or constraining forces to be spread over a relatively larger surface area of the stomach as compared to certain prior art devices. Spreading a force over a relatively larger surface area decreases the amount of force that any given point within that surface area experiences. Without being bound by any particular theory or mode of action, when this concept is applied to the stomach through devices and methods of the invention the constricted or constrained stomach is less likely to experience erosions, which can cause leaks in the stomach and result in emergency surgeries to repair the breach.

Yet another advantage of certain devices and methods of the present invention as described in detail herein is the use of relatively smooth contact points where the device interacts with the stomach. Certain devices of the prior art have relatively sharp or abrupt edges or device features that can act as focal points for forces on the stomach. Such focal points can lead to erosions. The various parts of certain embodiments of the invention are designed and configured to present rounded or “soft” edges and features to the stomach and the blood vessels in the area of the stomach so as to reduce the incidence of erosions and the compression or dissection of blood vessels and connective tissue.

Another advantage of certain embodiments of the present invention is that the device can conform to the stomach in such a way that the device moves with the stomach. When the device moves with the stomach it can substantially maintain its constrictive or constraining function even when the stomach moves. The conforming and adjustable nature of certain embodiments of the invention allows the device to move with the stomach. Optionally, the device may also be anchored to the stomach at one or more locations around the stomach using conventional anchoring techniques. Optionally, the device may also be anchored to the abdominal wall at one or more locations using conventional anchoring techniques.

According to certain embodiments of the invention, devices may be dimensioned and configured to be inserted using minimally invasive surgical techniques known the art. Devices may be inserted laproscopically, transorally, or through other means or procedures for accessing the extra-gastric abdominal space.

FIG. 2 illustrates a perspective view of device 100 according to certain embodiments of the invention. Device 100 includes expandable body 110 which has a helical configuration. According to certain embodiments of the invention, expandable body 110 has helical configuration when it is in its unexpanded condition. In such embodiments, that helical configuration of expandable body 110 has dimensions that facilitate its advancement around the stomach. While expandable body 110 has a helical configuration, it remains flexible such that it can be manipulated, bent, or otherwise contorted while being advanced about the stomach.

According to certain alternate embodiments of the invention, expandable body 110 is designed to assume a helical configuration upon at least partial expansion but does not have a helical configuration when it is in its unexpanded condition. In such embodiments, expandable body 110 is in a flexible condition in its unexpanded state which can facilitate its introduction into the abdominal space. Further, in such embodiments the flexible condition of expandable body 110 can facilitate its advancement and positioning around the stomach. In some embodiments the device is advanced and positioned around the stomach using a cannula, sheath, or other similar tool. The cannula or sheath is retracted when expandable body 110 is at or near its desired position about the stomach. Alternatively, a guide-wire-like device or a guide tube (which may include a flexible endoscope) with a blunt tip can used to establish the pathway around the stomach such that the Implant can be advanced and guided in place via the guide-wire.

Referring still to FIG. 2, according to certain embodiments of the invention device 100 includes connector 190. Connector 190 is in fluid communication with the internal volume of expandable body 100 and facilitates the expansion and adjustment of expandable body 100. Connector 190 has a length to allow a proximal end portion thereof to extend out of the body of the patient when device 100 is implanted in the desired location and orientation in the abdominal space of a patient and expanded. Connector 190 may run along the surface of expandable body 110 and may be free of the surface of expandable body 110 except for where it inserts through the wall of expandable body 110. According to certain alternate embodiments of the invention, connector 190 may be fixed at one or more locations along its length that is adjacent to expandable body 110, or the entire adjacent length may be fixed to the surface of expandable body 110, such as by adhesive, welding, vulcanization, taping, or other mechanical fixation.

Expandable body 110 can be expanded with gas or liquid or both. Examples of gases or liquids that can be used to expand expandable body 110 include, but are not limited to: carbon dioxide, helium, isotonic dextrose solution, isotonic saline solution, and air. Connector 190 allows for expandable body 110 to be adjusted at several time points after implantation. In certain embodiments, expandable body 110 is repeatedly adjusted over a course of several follow-up visits by the patient subsequent to implantation. In many cases, expandable body 110 is further expanded by adding more gas or liquid as the patient adapts to the device. Further expansion can be part of a method of treatment to gradually constrain or constrict the stomach by increasing amounts. In some embodiments, different regions of expandable body 110 can be further expanded by differing amounts. For example, a region of expandable body 110 near the fundus could be expanded to a greater degree than a region of expandable body 110 near another area of the stomach in a subsequent expansion step.

Referring still to FIG. 2, the helical configuration of expandable body 110 has dimensions appropriate for placing device 100 about the stomach. The inner circumference of the helical configuration ranges from about 30.5 cm (12 inches) to about 91.4 cm (36 inches), and is preferably from about 45.7 cm (18 inches) to about 71.1 cm (28 inches). The pitch of the helical configuration (or the space between the approximate centerline of a turn of the expandable body 110 and the next adjacent turn of the expandable body ranges from about 2.5 cm (1 inch) to about 15.2 cm (6 inches), and is preferably from about 5.1 cm (2 inches) to about 10.2 cm (4 inches).

According to certain embodiments of the invention, expandable body 110 is formed of silicone, preferably using a dip-molding or similar method. Alternatively, expandable body 110 may be formed by co-extrusion, e.g., co-extruding EVOH (ethylene-vinyl alcohol copolymer) and polyurethane. As another alternative embodiment, expandable body 110 may be formed of a blend of silicone and polyurethane. As a further alternative, expandable body 110 can be formed from or include one or more semi-compliant or non-compliant materials. Examples of useable semi-compliant materials include, but are not limited to: nylon, polyethylene, polyester, polyamide and polyurethane. Polyurethane, nylon, polyethylene and polyester can be compliant or semi-compliant materials, depending upon the specific formulation and hardness or durometer of the material as produced. Examples of noncompliant materials that can be used in the construction of expandable bodies described herein include, but are not limited to: polyethylene terepthalate (PET), polypropylene, poly ether-ether ketone, polysulfone and urethane.

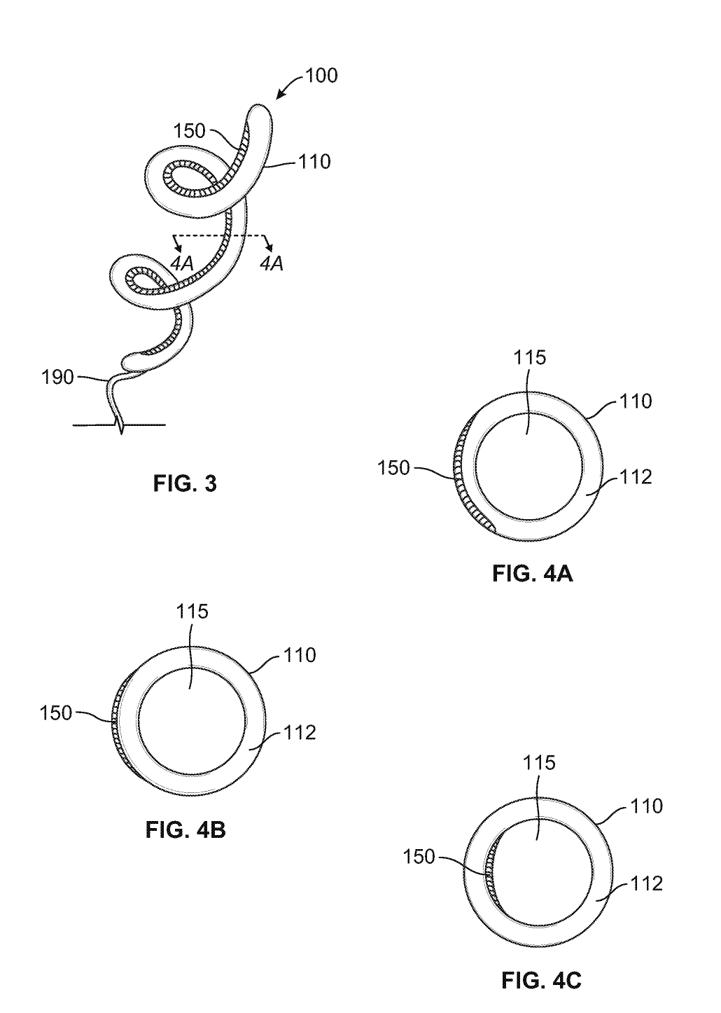

FIG. 3 illustrates a perspective view of device 100 having reinforced region 150 according to certain embodiments of the invention. FIG. 3 depicts reinforced region 150 extending along a substantial length of expandable body 110, but according to alternative embodiments of the invention reinforced region 150 may extend along more or less than the length depicted in FIG. 3. Further, FIG. 3 depicts reinforced region 150 as a single continuous region on expandable body 110, but according to alternative embodiments of the invention there may be a plurality of discontinuous reinforced regions. Alternatively, the reinforcement may be achieved by a thicker wall thickness in that region, but with the same material as the non-reinforced sections. Still further, FIG. 3 depicts reinforced region 150 as reinforcing the interior surface of the helical configuration of expandable body 110.

FIG. 4A illustrates a cross-sectional view of expandable body 110 including a cross-section of reinforced region 150. FIG. 4A illustrates expandable body 110 having expandable body wall 112 and expandable body internal volume 115. The cross-section of expandable body 110 has radial dimensions that refer to the distance from the surface of expandable body wall 112 which is in contact with the stomach to the outer surface of expandable body wall 112 which is directly opposite such an area of contact with the stomach. This radial dimension can be referred to as the radial thickness of expandable body 110. Expandable body 110 has a radial thickness in its unexpanded condition and a radial thickness it its expanded condition. In some embodiments, the radial thickness of expandable body 110 in its unexpanded condition ranges from about 0.25 cm (0.1 inches) to about 2.5 cm (1.0 inches). Preferably, the radial thickness of expandable body 110 in its unexpanded condition ranges from about 0.5 cm (0.2 inches) to about 1.2 cm (0.5 inches). In some embodiments, the radial thickness of expandable body 110 in its expanded condition ranges from about 1.2 cm (0.5 inches) to about 10 cm (4 inches). Preferably, the radial thickness of expandable body 110 in its expanded condition ranges from about 4 cm (1.6 inches) to about 8 cm (3 inches). In some embodiments, expandable body wall 112 has a thickness that ranges from about 0.12 cm (0.05 inches) to about 1.2 cm (0.5 inches). Preferably, expandable body wall 112 has a thickness that ranges from about 0.25 cm (0.1 inches) to about 0.6 cm (0.25 inches).

Referring still to FIG. 4A, reinforced region 150 is depicted as being located within expandable body wall 112, though preferentially located near the interior surface of the helical configuration of expandable body 110. According to embodiments of the invention where reinforced region 150 is located within expandable body wall 112, reinforced region 150 may be located anywhere along the radial thickness of expandable body wall 112 and not only towards the outermost region of radial thickness of expandable body wall 112 as depicted in FIG. 4A.

FIGS. 4B and 4C illustrate alternative embodiments of cross-sections of expandable body 110 including reinforced region 150 according to certain embodiments of the invention. It is understood that in both FIGS. 4B and 4C, reinforced region 150 is present on the interior surface of the helical configuration of expandable body 110 as depicted in FIG. 3. FIGS. 4B and 4C illustrate alternate ways of reinforcing the interior surface of the helical configuration of expandable body 110. FIG. 4B depicts an embodiment in which reinforced region 150 are located on the generally outermost region of the cross-section of expandable body wall 112. As compared to FIG. 4A, reinforced region 150 is not within expandable body wall 112 in the embodiment depicted in FIG. 4B. FIG. 4C depicts an embodiment in which reinforced region 150 is located on the generally innermost region of the cross-section of expandable body wall 112. As compared to FIG. 4A, reinforced region 150 is not within expandable body wall 112 in the embodiment depicted in FIG. 4C. According to certain embodiments of the invention, reinforced region 150 may be located such that it is within the outermost or innermost region of expandable body wall 112 and also extends radially beyond those outermost or innermost regions of expandable body wall 112.

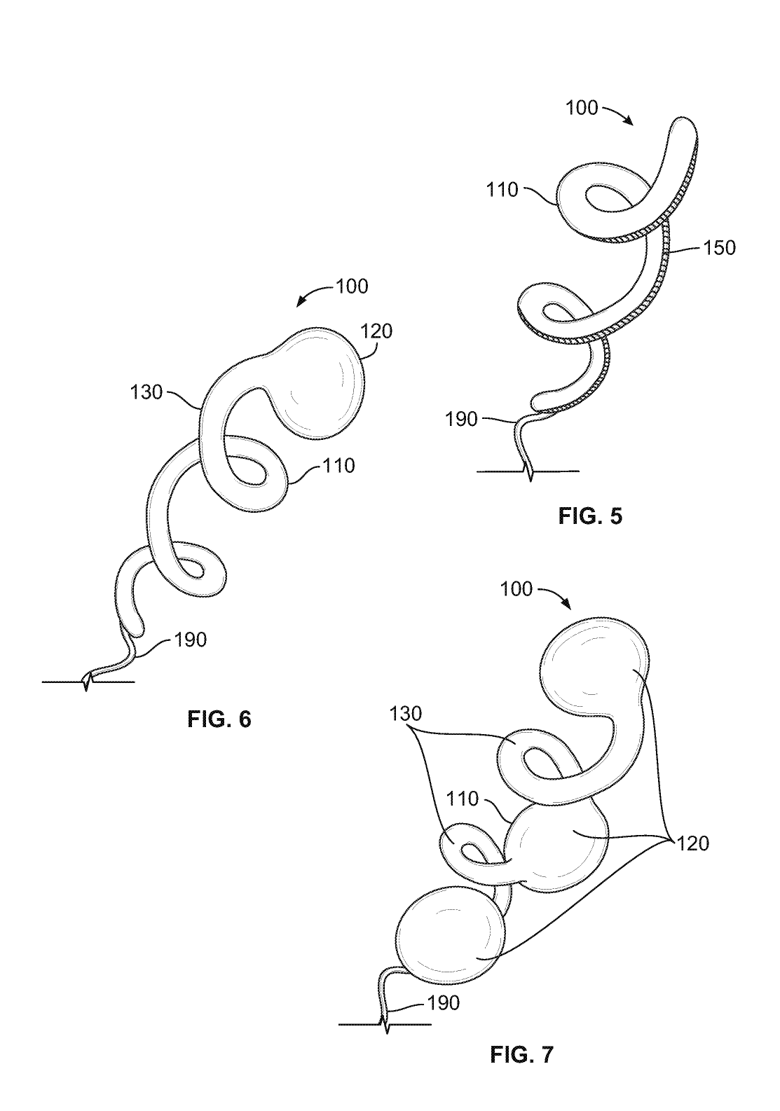

FIG. 5 illustrates a perspective view of device 100 having reinforced region 150 according to certain embodiments of the invention. In contrast to FIG. 3, FIG. 5 depicts reinforced region 150 as reinforcing the exterior surface of the helical configuration of expandable body 110. Similar to the embodiments depicted in FIGS. 4A, 4B, and 4C, reinforced region 150 can be located within expandable body wall 112, at the outermost or innermost surface of expandable body wall 112, and combinations thereof. Further, reinforced regions may be located at a plurality of areas of the cross-section of expandable body wall 112.

The reinforced regions of certain embodiments of the invention can resist expansion under conditions that cause some or all of the rest of the expandable body to expand. According to certain embodiments, the reinforced regions may alternately or additionally influence the direction of expansion of the expandable body under conditions that cause the rest of the expandable body to expand. Generally speaking, the expandable body will expand preferentially away from the reinforced region. According to certain embodiments, the reinforced regions may alternately or additionally facilitate the helical conformation of the expandable body when the expandable body is unexpanded, partially expanded, and/or substantially expanded. Generally speaking, in each of these embodiments and their combinations, such reinforced regions are less compliant than the region of the expandable body that they reinforce.

The reinforced regions can be made by any of the following methods or similar methods. In some embodiments, the reinforced regions are composed of multiple layers of material added to expandable body wall 112. The layers of material can be added to the exterior surface of the body wall or to the interior surface, or can be captured inside the wall thickness. In some embodiments, expandable body 110 is turned inside out to facilitate the addition of multilayer reinforced regions and then returned to its proper configuration. In other embodiments, multilayer reinforced regions are added to the interior surface of expandable body wall 112 through openings in expandable body 110 and such openings are subsequently sealed.

Although many different multilayer processes could be used to fabricate the reinforced regions, the following steps can produce a suitable multilayer reinforced region. Several layers of non-vulcanized polymer (e.g., non-vulcanized silicone) may be layered against the surface expandable body 110. A reinforcing layer of a polyester mesh or other low compliance material may be layered among the backing layers. These multiple layers may be laid up in a variety of arrangements to achieve a desired level of reinforcement balanced against the layer thickness and the flexibility of the expandable body. With the layers laid up in the preferred arrangement, pressure and heat are applied to vulcanize the non-vulcanized materials. The layers bond to form non-separable layers. As an example, the layers are vulcanized in a vulcanizing press at about 175 degrees C. and about 100 pounds per square inch (psi) pressure for about twenty minutes, although these temperatures, pressures, and times may vary.

In some embodiments, reinforced regions can be formed within expandable body wall 112. One method for forming reinforced regions within the expandable body wall is to include one or more reinforcing layers during the fabrication of the expandable body wall. For example, during one type of dip-molding process for fabricating the expandable body, a mandrel or other form for making the body is repeatedly dipped into a polymeric solution to build up the thickness of the expandable body wall. During one or more of such dipping steps, a reinforcing layer, such as but not limited to a polyester mesh or other low compliance material, is positioned on the outer surface of the uncompleted expandable body, Subsequent dipping steps increase the thickness of the expandable body wall and cover the reinforcing layer or layers. At the conclusion of the dipping steps, the expandable body can be cured, whether by vulcanization or other curing process, to for the expandable body having a reinforced region within the expandable body wall.

In some embodiments, reinforced regions can be formed by varying the wall thickness of certain regions of the expandable body. For example, during one type of dip-molding process for fabricating the expandable body, a mandrel or other form for making the body is repeatedly dipped into a polymeric solution to build up the thickness of the expandable body wall. During the dipping process, certain regions of the uncompleted expandable body are subject to further dipping while other regions are not. The regions of the expandable body that have been subject to more dipping steps than other regions will have a greater wall thickness. Such areas of greater wall thickness form the reinforced regions of the expandable body.

In certain embodiments, reinforced regions are formed using some or all of the above methods or combinations thereof. The reinforced regions of a single expandable body may be formed from one or more of the above methods, their equivalents, or combinations thereof.

FIG. 6 illustrates a perspective view of device 100 according to certain embodiments of the invention. FIG. 6 depicts expandable body 110 of device 100 as having lobed region 120 and elongated region 130. According to certain embodiments of the invention, lobed region 120 is a region of expandable device 120 that is capable of expanding to occupy more surface area of the stomach than elongated region 130. FIG. 6 depicts lobed region 120 as being located near one end of elongated body 110. According to alternate embodiments, device 100 is designed such that lobed region 120 is located at other places along elongated body 110. Without being bound by a particular theory of mode of action, it is believed that lobed region 120 can distribute constraining or constrictive forces to a greater degree than elongated region 130. Thus, device 100 can be designed with lobed region 120 placed at a region of the stomach where it is desirable to distribute constraining or constrictive forces. Further, lobed region 120 can constrain or constrict more surface area of the stomach than elongated region 130 and device 100 can be designed with lobed region 120 placed at a region of the stomach where it is desirable to constrain or constrict more surface area of the stomach.

FIG. 7 illustrates a perspective view of device 100 according to certain embodiments of the invention in which elongated body 110 has a plurality of lobed regions 120 and a plurality of elongated regions 130. FIG. 7 depicts three lobed regions 120 and two elongated region 130, but it is understood that device 100 may be designed such that expandable body 110 has more or fewer lobed regions and more or fewer elongated regions. Similarly, as with the embodiment depicted in FIG. 6, lobed regions 120 can be located at places along elongated body 110 other than those depicted in FIG. 7. In certain embodiments of the invention, a plurality of lobed regions can effectively distribute constraining or constrictive forces on the stomach and can constrain or constrict a substantial amount of surface area of the stomach.

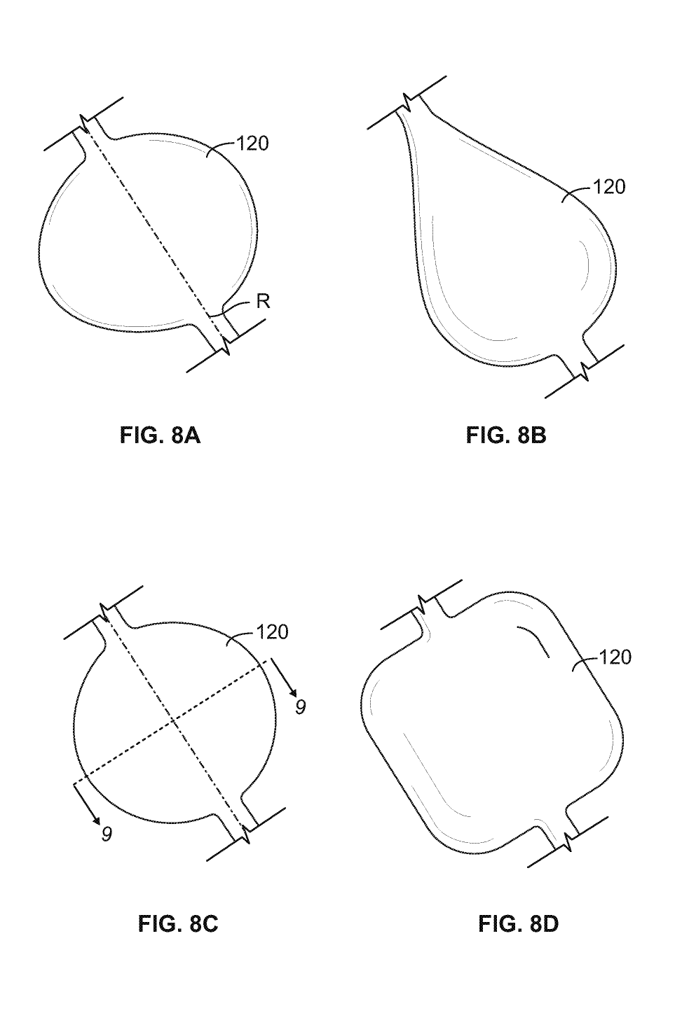

FIGS. 8A, 8B, 8C, and 8D illustrate plan views of various configurations of lobed region 120 according to certain embodiments of the invention. FIG. 8A depicts lobed region 120 as having an asymmetrical shape with respect to a reference line R which refers to the midpoint of the inlet and outlet of lobed region 120. FIG. 8B depicts lobed region 120 as having a teardrop shape. FIG. 8C depicts lobed region 120 a having a generally elliptical shape which is symmetric about the inlet and outlet of lobed region 120. FIG. 8D depicts lobed region 120 as having a generally rectangular shape, although the corner of the generally rectangular shape are rounded so that lobed region 120 presents atraumatic surfaces to the stomach and surrounding tissue. The shapes depicted in FIG. 8A through 8D are a small set of the possible shapes of lobed region 120 and it is understood that other shapes are within the scope of the invention. Further, an expandable body may have lobed regions of varying shapes and sizes, such shapes and sizes being chosen to facilitate effectively distribution of constraining or constrictive forces on the stomach.

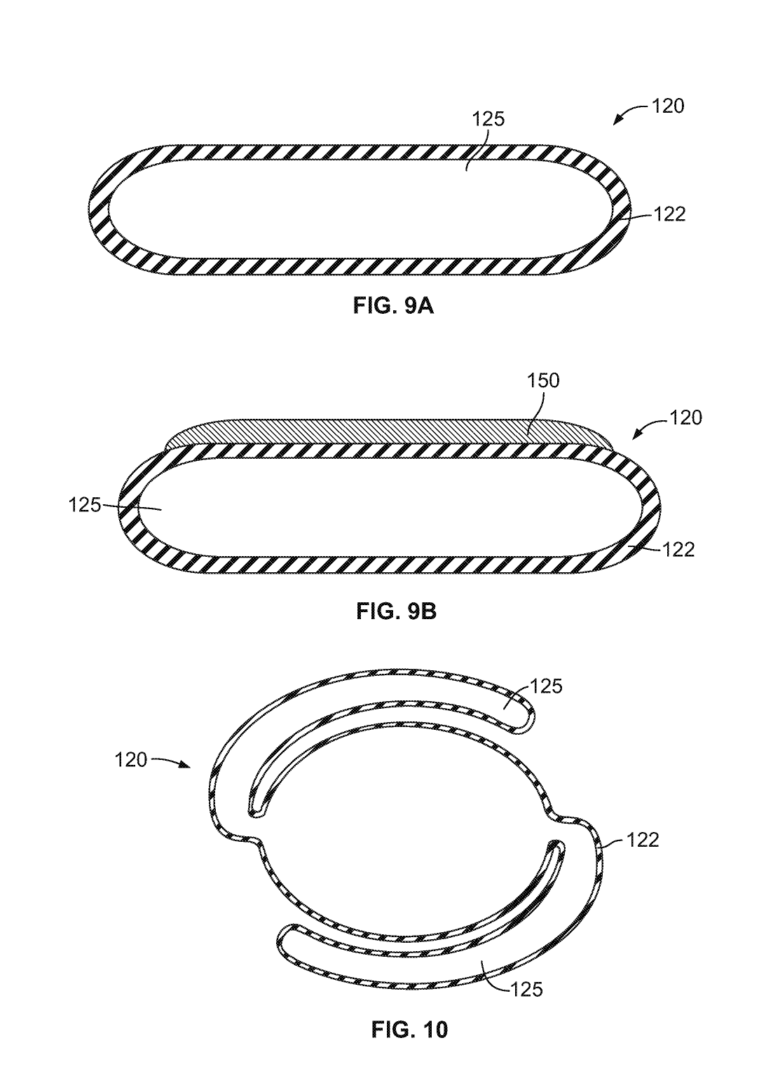

FIG. 9A illustrates a cross-sectional view of lobed region 120 according to the embodiment depicted in FIG. 8C. FIG. 9A depicts lobed region 120 as having lobed region wall 122 and lobed region internal volume 125. FIG. 9A depicts lobed region 120 in an expanded configuration. In certain embodiments, when in its unexpanded configuration, lobed region 120 has cross-sectional dimensions similar to the elongated region or regions of device 100, such as those depicted in FIGS. 4A, 4B, and 4C. According to certain embodiments of the invention, some or all of lobed region wall 122 stretches such that lobed region 120 can expand into its lobed configuration.

FIG. 9B illustrates a cross-sectional view of lobed region 120 in which lobed region 120 has reinforced region 150 according to certain embodiments of the invention. FIG. 9B depicts reinforced region 150 as being located at the outermost surface of lobed region wall 122. Similar to the embodiments depicted in FIGS. 4A, 4B, and 4C, reinforced region 150 can be located within lobed region wall 122, at the outermost or innermost surface of lobed region wall 122, and combinations thereof. Further, reinforced regions may be located at a plurality of areas of the cross-section of lobed region wall 122. It is understood that, the identity of these reinforced regions is consistent with the previous discussion of reinforced regions and that these reinforced regions may alternately or additionally influence the direction of expansion of the lobed regions under conditions that cause the rest of the lobed region to expand.

One of the novel aspects of certain embodiments of the invention is the configuration of the lobed regions. The lobed regions are dimensioned and configured to occupy a relatively large surface area of the stomach without occupying a commensurately large volume of space in the abdominal cavity. For example, as compared to an expandable device of the prior art having a generally spherical cross-section, the lobed regions described herein occupy less abdominal volume while occupying a similar or greater surface area of the stomach. As compared to prior art expandable devices, lobed regions of certain embodiments of the invention can be said to have a low profile. Lobed regions of certain embodiments have a higher ratio of stomach-contacting surface area to internal volume than certain prior art expandable devices. The low profile of the expanded device and relatively high surface area to internal volume ratios can facilitate the distribution of constraining or constricting forces about the stomach while reducing the compression of, dissection or, or other trauma to the surrounding blood vessels and connective tissue.

In some embodiments, lobed regions of a single expandable body may have the same surface area to internal volume ratio, the lobed regions may have different ratios, or some of the lobed regions may have the same ratio and some may have different ratios. In embodiments, where the expandable body has lobed regions of varying shapes and sizes, the surface area to volume ratios may be the same or different. That is, a lobed region with a given shape may have the same surface area to volume ratio as a lobed region with a different shape. Or, the ratio of differently-shaped lobed regions may be different.

For the lobed regions, the surface area of the lobed region that is in contact with the stomach can range from about 23 cm² (3.5 square inches) to about 300 cm² (42 square inches). Preferably, the surface area of the lobed region that is in contact with the stomach can range from about 50 cm² (8 square inches) to about 138 cm² (22 square inches). The internal volume of the lobed region can range from about 15 cm³ (1 cubic inch) to about 525 cm³ (32 cubic inches). Preferably, the internal volume of the lobed region can range from about 50 cm³ (3 cubic inches) to about 215 cm³ (13 cubic inches). In certain embodiments, the ratio of the surface area of the lobed region that is in contact with the stomach to the internal volume of the lobed region can range from about 20:1 to about 0.6:1. Preferably, ratio of the surface area to the internal volume of the lobed region can range from about 6:1 to about 1.5:1.

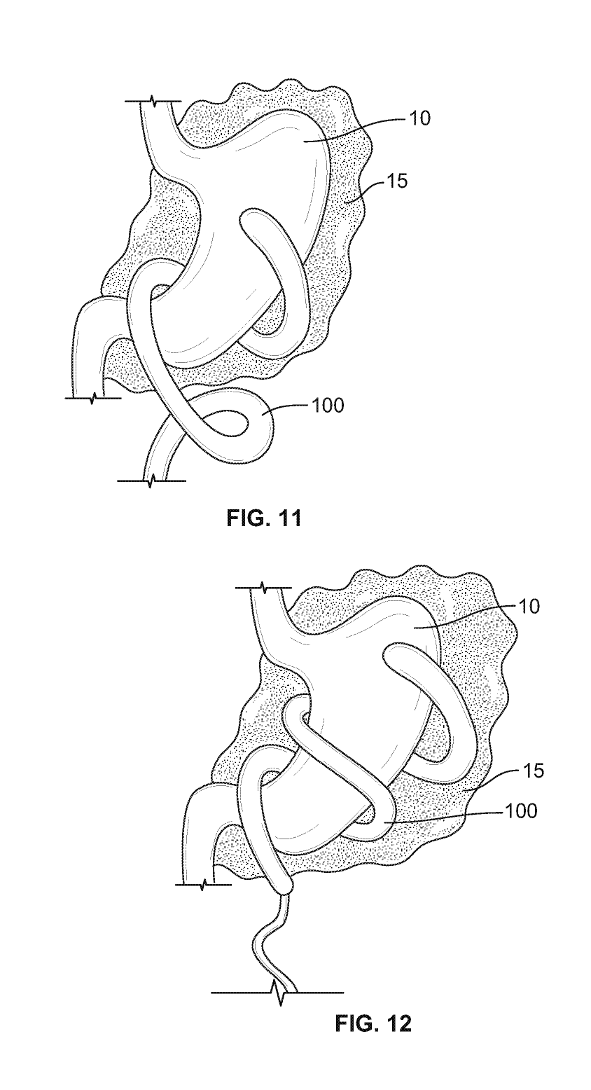

FIG. 10 illustrates a cross-sectional view of a device in which an unexpanded lobed region 120 has areas wrapped about the region according to certain embodiments of the invention. FIG. 10 illustrates lobed region 120 having lobed region wall 122 which enclosed lobed region internal volume 125. In unexpanded configuration, lobed region 120 has wing regions that can be wrapped, twisted, or compressed against the central axis of lobed region 120 to form a low profile device. In such a low profile condition, the wrapped and unexpanded (or slightly expanded) lobed region can be placed inside of a cannula or retractable sheath and advanced and positioned about the stomach. The cannula or sheath can be removed and the lobed region expanded such that the wings unwrap, untwist, or decompress and form the lobed region. Any of the shapes presented in FIGS. 8A thought 8D, or any other shapes suitable for the lobed region, can be formed in such a winged configuration.

In general, the lobed regions can be formed using any of the methods discussed for forming the expandable body and its reinforced regions. In certain embodiments, the reinforced regions are on or near the exterior surface of the lobed region, making the exterior surface less compliant than the interior surface of the lobed region. Again, the terms exterior and interior refer to the general helical configuration of the expandable body such that the interior surface of the helix and the lobed region thereon is the surface designed to be in contact with the stomach. In such a configuration, the less compliant exterior surface of the lobed region will resist expansion more than the interior surface of the lobed region. The more compliant interior surface will expand away from the less compliant exterior surface and exert force on the stomach. In certain embodiments, the interior surface of the lobed region expands such that the lobed region juts into or expands into the normal shape of the stomach. In certain embodiments, one or more lobed regions expand such that their expansion distends the stomach wall inward to reduce the overall stomach volume instead of or in addition to the reduction in overall stomach volume attained by the expandable body providing constraining or constrictive forces. For example, a lobed region placed near the fundus may preferably be designed to jut into or expand into the stomach wall in that region such that the volume of the stomach near the fundus is decreased comparatively more than other regions of the stomach. Similarly, a lobed region placed near the fundus may exert comparatively greater force on the fundus region of the stomach by jutting into, expanding into, or otherwise distending the stomach wall in the fundus region.

In certain embodiments, the expandable body is fabricated using a dip-molding process or other process that allows for selective control over the regional wall thickness of the expandable body (e.g., liquid injection molding, blow molding, rotational molding). In such embodiments, elongated regions can be made to have greater wall thickness than lobed regions. The greater wall thicknesses in the elongated regions will make the elongated regions less compliant. The lobed regions, with comparatively thinner walls, will expand more than the less compliant elongated regions. Such a configuration where one or more elongated region has a greater wall thickness than one or more lobed region can be combined with the other methods disclosed herein for creating reinforced regions.

FIG. 11 illustrates a perspective view of device 100 being advanced around stomach 10 according to certain embodiments of the invention. In FIG. 11, the blood vessels and connective tissue located along or nearby the inner curvature and outer curvature of stomach 10 are generally represented by abdominal region 15. While device 100 is being advanced around stomach 10, care is taken to reduce or avoid trauma to vessels and tissues in abdominal region 15. The helical configuration of device 100 can facilitate the advancement of device 100. Where dissections in connective tissue are necessary, the relatively small cross-section of the unexpanded or partially expanded device 100 can further facilitate the advancement of device 100 while reducing or avoiding vessel or tissue trauma.

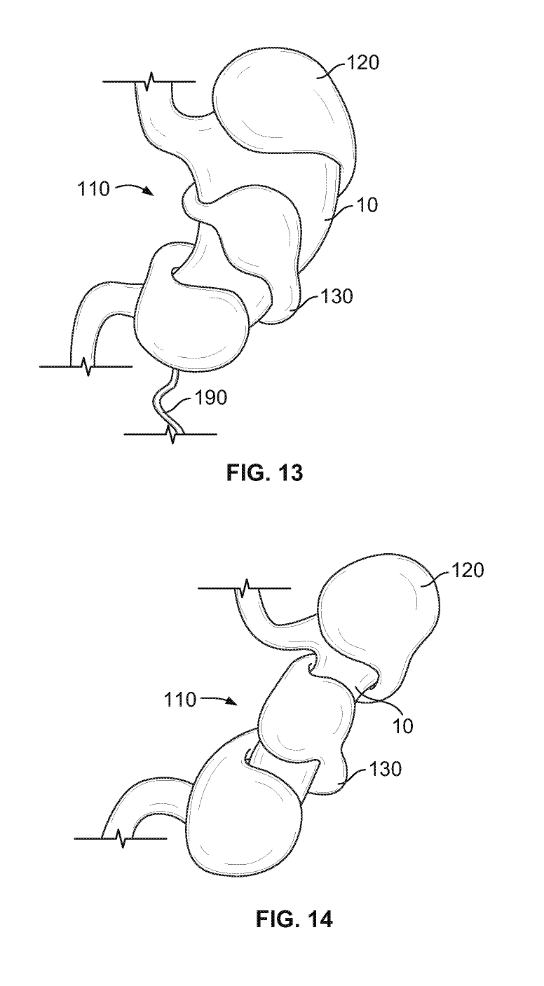

FIG. 12 illustrates a perspective view device 100 being advanced around stomach 10 such that a region of device 100 is positioned near the fundus according to certain embodiments of the invention. In such embodiments, positioning device 100 near the fundus can facilitate compression and constriction of the fundus while also enhancing the ability of device 100 to stay in place relative to stomach 10. Relative motion between device 100 and stomach 10 has potential complications in situations where the relative motion is focused on a particular part of stomach 10 and/or when the relative motion is accompanied by pressure focused on a particular part of stomach 10. For example, such motion between the device and stomach can cause erosions of the stomach wall if there is focused motion and/or focused pressure. As another example, motion between the device and stomach can displace the device relative to the stomach such that the device no longer delivers the desired constrictive or compressive forces.

FIG. 13 illustrates a perspective view of lobed regions 120 of device 100 being expanded after device 100 is positioned about stomach 10 according to certain embodiments of the invention. FIG. 13 depicts lobed regions 120 positioned near the fundus of stomach 10, at an approximate midpoint of stomach 10, and near the pylorus of stomach 10. In certain embodiments, positioning device 100 near one or both end regions of stomach 10 (such as near the fundus or pylorus) can facilitate compression and constriction of stomach 10 while also enhancing the ability of device 100 to stay in place relative to stomach 10. Further, the size and/or shape of lobed regions 120 may facilitate the atraumatic compression or constriction of stomach 10.

FIG. 13 depicts all of lobed regions 120 as positioned generally anterior to stomach 10 and all of elongate regions 130 positioned generally posterior to stomach according to certain embodiments of the invention. Such embodiments may enable stomach to be constrained or constricted while occupying relatively little space in the abdominal cavity and disrupting relatively little connective tissue posterior to the stomach. It is understood that, although FIG. 13 depicts all of lobed regions 120 as positioned on the same general side of stomach 10, expandable body 110 may be configured and/or positioned such that lobed regions 120 are not all on the same general side of the stomach. Expandable body 110 may be configured and/or positioned such that lobed regions 120 are distributed around the body in any number of orientations.

FIG. 14 illustrated a perspective view of device 100 compressing stomach 10 according to certain embodiments of the invention. In such embodiments, lobed regions 120 and elongate regions 130 of expandable body 110 are expanded to an extent that provides stable and relatively uniform compression of stomach 10.

The device of embodiments of the invention preferably moves with the stomach even though it is not physically anchored to the stomach by staples, sutures, or other conventional means. Of course, it is understood that such convention means of anchoring are within the scope of the invention. Preferably, the device remains in contact with the stomach whether the stomach is empty, full, or some state in between. Maintaining contact with the stomach is preferable to prevent the stomach from migrating away from the device and to prevent or decrease the incidence of erosions or other irritation of the stomach wall as compared to prior art devices. The helical shape of certain embodiments of the device is designed to fit about the stomach regardless of the presence of food in the stomach. The expandability of the lobed regions of the device, the presence of reinforcing regions of the device, and the pitch and radial dimensions of the helical configuration are chosen to maintain contact with the stomach.

Although the devices of certain embodiments of the invention are effectively anchored to the stomach by virtue of their dimensions and configuration, it is understood that conventional physical anchors that bond with or otherwise physically interact with the stomach or the abdominal wall may optionally be used to further anchor the device to the stomach or the abdominal wall, in particular the anterior abdominal wall. For example, tissue ingrowth meshes can be included on or near a surface of the device. An ingrowth layer (preferably, but not necessarily made of a layer of velour, such as a polyester fiber configured and dimensioned to encourage tissue growth into it) is laid on the outside surface of the expandable body. Multiple ingrowth layers can be used, each having a relatively less dense or more dense weave than the first ingrowth layer. The layers can be bonded to the expandable body using the multilayer vulcanization techniques discussed herein or other equivalent bonding techniques, including adhesive bonding and suturing. The tissue ingrowth mesh can be sutured to the abdominal wall (and/or optionally to the stomach) to anchor the device. By anchoring the device, which is fitted close to the stomach, the stomach is thereby also fixed in place as compared to an unanchored stomach.

Attachments can also be utilized to attach a section of the device to another section of the device. For example, as the device wraps around the stomach, each section of the device on the anterior side of the stomach can be attached or linked to its adjacent section of the device that is also on the anterior side of the stomach. This would link the sections of the helix together and achieve a more complete constraint of the stomach. An attachment or link could be accomplished with a silicone to silicone mechanical join such as a silicone tubing being threaded through a silicone loop, or by another configuration.

One advantage of embodiments of the inventions is that these devices can provide significant coverage of the surface area of the stomach after they have been advanced and positioned about the stomach. However, as compared to other device designs, devices dimensioned and configured according to embodiments of the invention can be advanced and positioned while causing comparatively less trauma to the blood vessels and tissue on or near the stomach. The comparatively small cross-section of the unexpanded device allows it to be positioned without significant trauma to blood vessels and tissue. Then, after positioning, the lobed regions can be expanded to provide the compressive and constrictive forces of a comparatively larger device. The expansion of the lobed regions once they are in position can be done while causing comparatively less trauma than a larger device. Further, the low profile and high surface area to volume ratio of the lobed regions can also enable the device to provide the compressive and constrictive forces while causing comparatively less trauma than prior art devices.

While the invention has been described with reference to certain embodiments, it will be understood by those skilled in the art that various changes may be made and equivalents may be substituted without departing from the scope of the invention. In addition, many modifications may be made to adapt a particular situation or material to the teachings of the invention without departing from its scope. Therefore, it is intended that the invention not be limited to the particular embodiment disclosed, but that the invention will include all embodiments falling within the scope of the appended claims.

1. A device for the treatment of obesity, the device comprising:
an expandable body having a helical configuration suitable for being positioned about the exterior of the stomach of a patient, the body comprising a first region having an elongated configuration when expanded and a second region having a lobed configuration when expanded.

2. The device of claim 1 wherein the body further comprises a first plurality of regions each having an elongated configuration when expanded and a second plurality of regions each having a lobed configuration when expanded.

3. The device of claim 1 wherein the first region and the second region have substantially the same cross-section when unexpanded.

4. The device of claim 1 wherein first region and the second region have substantially the same cross-section when partially expanded.

5. The device of claim 1 wherein the lobed configuration is a disk-like configuration.

6. The device of claim 1 wherein the body further comprises a region reinforcing an interior surface of the helical configuration.

7. The device of claim 1 wherein the body further comprises a region reinforcing an exterior surface of the helical configuration.

8. The device of claim 1 wherein the body further comprises a region reinforcing an exterior surface of the second region.

9. The device of claim 1 wherein the body further comprises an ingrowth layers for attaching the device to the abdominal wall.

10. A method for the treatment of obesity, the method comprising:
accessing an extra-gastric abdominal space of a patient;
introducing an expandable device into the extra-gastric abdominal space, the expandable device comprising a first region having an elongated configuration when expanded and a second region having a lobed configuration when expanded wherein the expandable device has a helical configuration suitable for being positioned about the exterior of the stomach;
advancing the expandable device around the stomach; and
expanding the first region and the second region to constrain the stomach.

11. The method of claim 10 wherein the expandable device comprises a first plurality of regions each having an elongated configuration when expanded and a second plurality of regions each having a lobed configuration when expanded.

12. The method of claim 11 further comprising positioning the device about the stomach wherein the second plurality of regions are each substantially anterior to the stomach.

13. The method of claim 11 further comprising positioning the device wherein none of the second plurality of regions are substantially posterior to the stomach.

14. The method of claim 10 further comprising positioning the device about the stomach wherein upon expansion the second region constrains the fundus.

15. The method of claim 10 wherein the second region has a range of lobed configurations that vary with the extent of expansion of the second region and further comprising expanding the second region to less than full extent.

16. A device for the treatment of obesity, the device comprising:
an expandable body having a helical configuration suitable for being positioned about the exterior of the stomach of a patient, the body comprising at least one expandable region having surface area to internal volume ratio of greater than about 1.5:1 when expanded.

17. The device of claim 16 wherein the body further comprises at least one expandable region having an elongated configuration.

18. The device of claim 16 wherein the body further comprises a plurality of expandable regions having surface area to internal volume ratio of greater than about 1.5:1 when expanded.

19. The device of claim 18 wherein the at least one of the plurality of expandable regions has a surface area to internal volume ratio different from another of the plurality of expandable regions when expanded.

20. The device of claim 16 wherein the body further comprises a region reinforcing an exterior surface of the helical configuration.
